(19) United States
(12) Patent Application Publication
(10) Pub. No.: US 2003/0163118 A1
(43) Pub. Date: Aug. 28, 2003

(54) Catheter having a tapered distal tip and method of making

(76) Inventors: Rasean L. Hamilton, Santa Clara, CA (US); Cheryl Rice, San Diego, CA (US); Sandra K. Schenk, San Jose, CA (US); Arthur J. Wen, Sunnyvale, CA (US); Anh D. Tran, Sunnyvale, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 09/577,217

(22) Filed: May 23, 2000

(51) Int. Cl. A61M 25/00

(52) U.S. Cl. 604/525; 604/96.01; 264/632

(57) ABSTRACT

An intraluminal catheter comprising an elongated shaft having a proximal end, a distal end, at least one lumen, and a distal tip having an internal taper. The distal tip has an inner surface tapering distally to an inner diameter smaller than an inner diameter of the shaft proximal to the tapered distal tip. In one embodiment, the distal tip includes an outer surface tapering distally to an outer diameter smaller than an outer diameter of the shaft proximal to the tapered distal tip. In a presently preferred embodiment, the distal tip defines at least in part a guidewire receiving lumen. A method of making a catheter shaft section with an internal taper in accordance with the invention generally includes placing at least a section of a polymeric tube having a first end, a second end and a lumen therein on a section of a mandrel having a straight cylindrical surface with a substantially constant outer diameter, so that a gap exists between an inner surface of the polymeric tube and an outer surface of the mandrel. The polymeric tube on the mandrel is placed within a mold having a tapered end. The polymeric tube is heated and the first end of the polymeric tube is placed in contact with the tapered end of the mold, so that an internal taper is formed in the first end of the polymeric tube.

### BACKGROUND OF THE INVENTION

This invention generally relates to medical devices, and particularly to intraluminal catheters.

In percutaneous transluminal coronary angioplasty (PTCA) procedures, a guiding catheter is advanced until the distal tip of the guiding catheter is seated in the ostium of a desired coronary artery. A guidewire, positioned within an inner lumen of a dilatation catheter, is first advanced out of the distal end of the guiding catheter into the patient's coronary artery until the distal end of the guidewire crosses a lesion to be dilated. Then the dilatation catheter having an inflatable balloon on the distal portion thereof is advanced into the patient's coronary anatomy, over the previously introduced guidewire, until the balloon of the dilatation catheter is properly positioned across the lesion. Once properly positioned, the dilatation balloon is inflated with liquid one or more times to a predetermined size at relatively high pressures (e.g. greater than 8 atmospheres) so that the stenosis is compressed against the arterial wall and the wall expanded to open up the passageway. Generally, the inflated diameter of the balloon is approximately the same diameter as the native diameter of the body lumen being dilated so as to complete the dilatation but not overexpand the artery wall. Substantial, uncontrolled expansion of the balloon against the vessel wall can cause trauma to the vessel wall. After the balloon is finally deflated, blood flow resumes through the dilated artery and the dilatation catheter can be removed therefrom.

In such angioplasty procedures, there may be restenosis of the artery, i.e. reformation of the arterial blockage, which necessitates either another angioplasty procedure, or some other method of repairing or strengthening the dilated area. To reduce the restenosis rate and to strengthen the dilated area, physicians frequently implant an intravascular prosthesis, generally called a stent, inside the artery at the site of the lesion. Stents are usually delivered to a desired location within a coronary artery in a contracted condition on a balloon of a catheter which is similar in many respects to a balloon angioplasty catheter, and expanded to a larger diameter by expansion of the balloon. The balloon is deflated to remove the catheter and the stent left in place within the artery at the site of the dilated lesion.

An outer surface of the distal end or tip of the catheter shaft is typically tapered, to lower the profile of the distal tip and facilitate advancement of the catheter in narrow vessels and across vessel occlusions. One difficulty has been the hanging up of the catheter distal tip on lesions or stent struts, wherein the distal tip folds back on itself after hitting the leading edge of the lesion or stent during advancement therethrough.

What has been needed is a catheter having an improved tapered distal tip.

### SUMMARY OF THE INVENTION

This invention is directed to an intraluminal catheter having an elongated shaft with a proximal end, a distal end, at least one lumen, and an internal taper in a distal section leading to the distal end. In a presently preferred embodiment, the internal taper is in a distal tip which defines at least in part a guidewire receiving lumen. The distal tip has an inner surface tapering distally to an inner diameter smaller than an inner diameter of the shaft proximal to the tapered distal tip. In one embodiment, the distal tip includes an outer surface tapering distally to a smaller outer diameter.

The distal tip internal taper decreases the inner diameter of the distal tip to thereby provide little or no gap between an inner surface of the distal tip and an outer surface of a guidewire disposed therein. The distal tip inner surface is sufficiently close to the guidewire to “hug” the guidewire and thereby prevent or inhibit the catheter distal end from hanging up on lesions or stent struts during advancement of the catheter in a patient's vasculature. In a presently preferred embodiment, a small gap is provided between the distal tip inner surface and the guidewire, so that the physician does not feel the guidewire touching the inner surface of the distal tip during longitudinal displacement of the guidewire and catheter relative to one another.

In one embodiment, the catheter is a balloon catheter generally including an elongated shaft having a distal tip with an internal taper and a balloon secured to a distal portion of the shaft. In one embodiment, the balloon catheter shaft comprises an outer tubular member defining an inflation lumen, and an inner tubular member having a guidewire lumen therein and the tapered distal tip having an internal taper on a distal end of the inner tubular member.

One embodiment comprises an assembly of a catheter, which in accordance with the invention has the distal tip with an internal taper, and a guidewire disposed within the lumen of the distal tip. The distal tip defines the distal end of a guidewire receiving lumen extending within at least a distal portion of the catheter, and the guidewire is slidably disposed within the guidewire receiving lumen. The distal end of the guidewire lumen defined by the distal tip has a relatively small inner diameter configured to allow longitudinal displacement of the guidewire therein and to restrain radial displacement of the guidewire therein.

A method of making a catheter shaft section with an internal taper in accordance with the invention generally includes placing at least a section of a polymeric tube having a first end, a second end and a lumen therein on a section of a mandrel having a straight cylindrical surface with a substantially constant outer diameter, so that a gap exists between an inner surface of the polymeric tube and an outer surface of the mandrel. The polymeric tube on the mandrel is placed within a mold having a tapered end. The polymeric tube is heated and the first end of the polymeric tube is placed in contact with the tapered end of the mold, so that an internal taper is formed in the first end of the polymeric tube.

The catheter of the invention has excellent crossability due to the tapered distal tip. As a result of the internal taper at the distal end of the catheter, radial displacement, or play, of the guidewire within the guidewire lumen is minimized, and preferably without adversely affecting longitudinal movement of the guidewire. Consequently, the internal taper in the distal tip provides improved catheter centering and prevents or inhibits the distal tip from hanging up on lesions or stent struts.

These and other advantages of the invention will become more apparent from the following detailed description when taken in conjunction with the accompanying exemplary drawings.

### BRIEF DESCRIPTION OF THE DRAWINGS

FIG. 1 is an elevational view, partially in section, of a balloon catheter which embodies features of the invention, having a distal tip with an internal taper.

FIG. 2 is an enlarged longitudinal cross sectional view of the catheter shown in FIG. 1, taken along line 2-2.

FIG. 3 is a transverse cross-section of the catheter shown in FIG. 2, taken at line 3-3.

FIG. 4 is a transverse cross-section of the catheter shown in FIG. 2, taken at line 4-4.

FIG. 5 is longitudinal cross sectional view of a tapered mandrel useful in a method of forming a catheter shaft with an internal taper which embodies features of the invention.

FIG. 6 is longitudinal cross sectional view of mold and mandrel, useful in a method of forming a catheter shaft with an internal taper which embodies features of the invention.

FIG. 7 is an enlarged longitudinal cross sectional view of a distal end of alternative embodiment of a catheter which embodies features of the invention, having a sleeve with a tapered outer surface on the inner tubular member.

FIG. 8 is longitudinal cross sectional view of mold and mandrel, useful in a method of forming a catheter shaft with an internal taper and a sleeve with a tapered outer surface which embodies features of the invention.

### DETAILED DESCRIPTION OF THE INVENTION

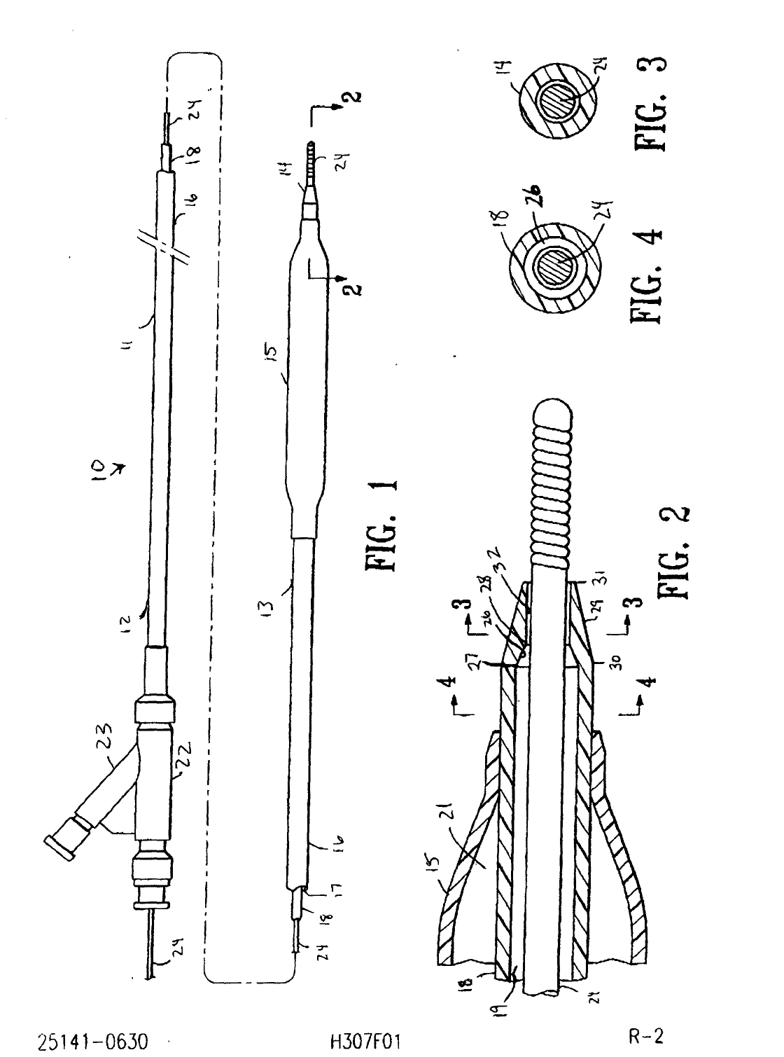

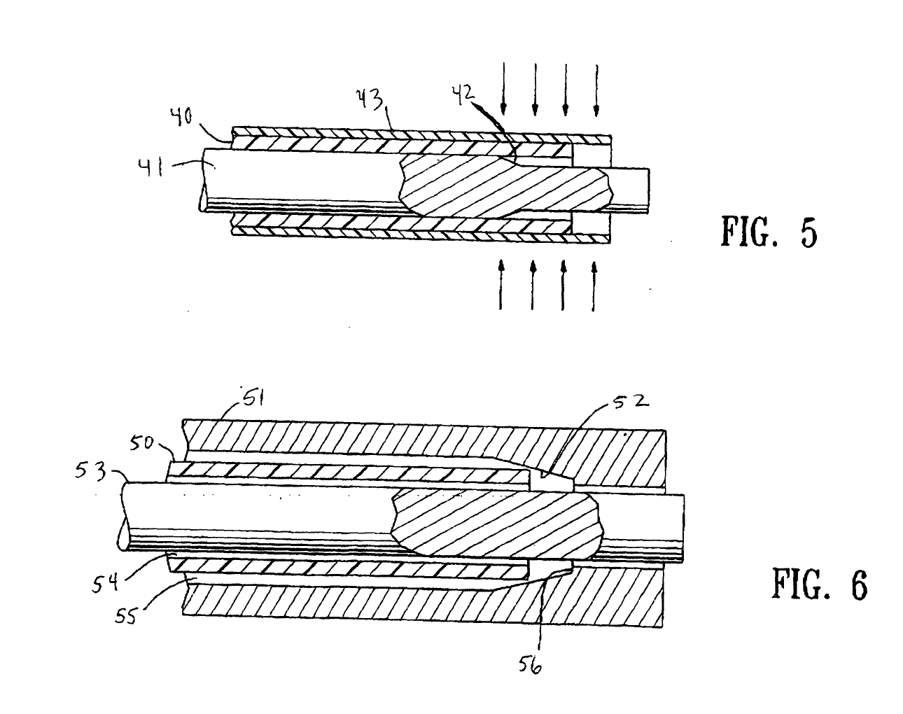

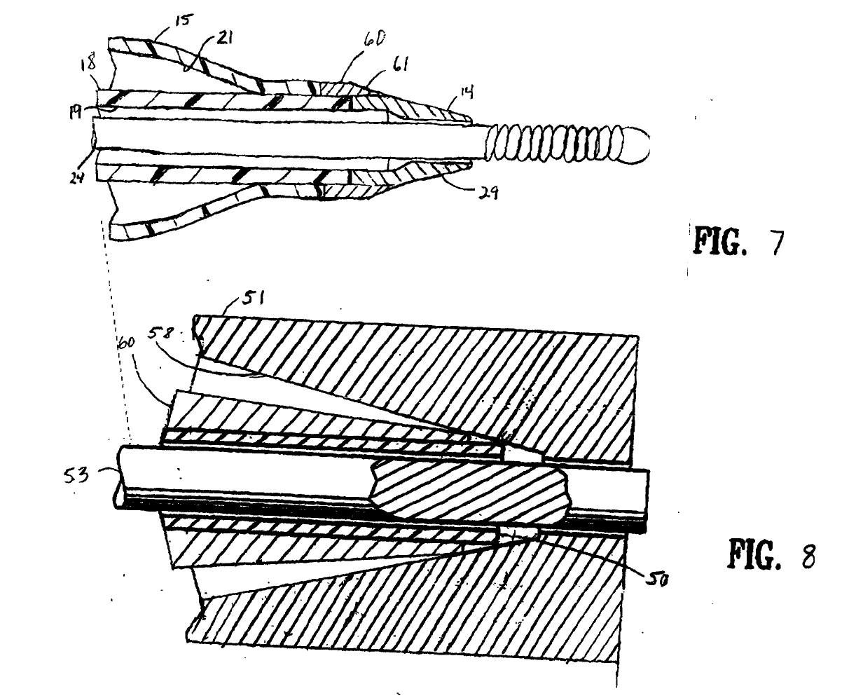

FIG. 1 illustrates an intraluminal catheter 10 which embodies features of the invention, generally comprising an elongated shaft 11 having a proximal end 12, a distal end 13, and a distal tip 14 having an internal taper. Catheter 10 is a balloon catheter having a balloon 15 secured to a distal portion of the shaft 11. In the embodiment illustrated in FIG. 1, the shaft 11 comprises an outer tubular member 16 defining an inflation lumen 17, and an inner tubular member 18 disposed within the outer tubular member and defining a guidewire lumen 19 configured to slidably receive a guidewire 24. In the illustrated embodiment, the coaxial relationship between outer tubular member 16 and inner tubular member 18 defines annular inflation lumen 17. Balloon 15 has a proximal end sealingly secured to the distal end of outer tubular member 16 and a distal end sealingly secured to the distal end of inner tubular member 18, so that its inflatable interior 21 is in fluid communication with inflation lumen 17. Adapter 22 at the proximal end of the shaft 11 is configured to direct inflation fluid through arm 23 into inflation lumen 17, and provide access to guidewire lumen 19.

As best illustrated in FIG. 2, showing an enlarged longitudinal cross section of a distal portion of the catheter 10 shown in FIG. 1, taken along lines 2-2, distal tip 14 is at the distal end of the inner tubular member 18 and defines in part the guidewire lumen 19. An internal taper 26, having a proximal end 27 and a distal end 28, is at the distal end of the distal tip 14, and has an inner surface tapering distally to an inner diameter which is smaller than an inner diameter of the inner tubular member 18 proximal to the distal tip 14. In the embodiment illustrated in FIG. 2, distal tip 14 has a tapered outer surface 29 tapering distally to an outer diameter smaller than an outer diameter of the inner tubular member 18 proximal to the distal tip 14. The tapered outer surface 29 tapers distally to the distal end of the catheter shaft 11. The tapered outer surface 29 is coextensive with at least a section of the internal taper 26, and in the embodiment illustrated in FIG. 2, proximal end 27 of the internal taper is radially aligned with a proximal end 30 of the tapered outer surface 29. In alternative embodiments, the proximal end 27 of the internal taper is located distal or proximal to the proximal end of the tapered outer surface 29 of the distal tip 14 (not shown). The distal end 28 of the internal taper 26 is longitudinally aligned with a distal end 31 of the tapered outer surface of the distal tip 14. However, in alternative embodiments, depending on the dimensions of the distal tip and the mold and mandrel used to form the distal tip, the distal end 28 of the internal taper 26 is not longitudinally aligned with a distal end 31 of the tapered outer surface of the distal tip 14 (not shown). The proximal end of the internal taper and the proximal end of the tapered outer surface of the distal tip 14 are typically distal to the proximal end of the distal tip 14, so that the proximal end of the distal tip 14 has an outer surface and an inner surface extending generally longitudinally aligned with the longitudinal axis of the distal tip 14. Section 32 extends between the distal end 28 of the internal taper 26 and the distal end 31 of the tapered outer surface, and defines a reduced inner diameter section of the distal end of the guidewire lumen 19. The outer surface preferably tapers at the same angle as or a greater angle than the angle of the internal taper 26, depending on the polymeric material used. Preferably, the outer surface of the distal tip tapers at an angel which is not greater than the angle of the internal taper, to minimize the wall thickness of the tapered section.

The distal end of guidewire lumen 19 has an inner diameter, due to the internal taper 26, which is configured to allow longitudinal displacement of a guidewire 24 within the guidewire lumen 19 and to restrain radial displacement of the distal end of guidewire 24 therein. In the embodiment illustrated in FIG. 2, a gap is provided between an outer surface of the guidewire 24 and the inner surface of section 32 of the distal tip 14. In a presently preferred embodiment, the gap is sufficient to allow free longitudinal displacement of the guidewire 24 within the guidewire lumen without frictional engagement of the guidewire 24 centered within the guidewire lumen. The inner diameter of the distal end of the distal tip 14 is about 3% to about 14%, preferably about 7% to about 11% larger than the outer diameter of the guidewire 24 disposed therein. For a guidewire having an outer diameter of about 0.34 mm to about 0.37 mm, the inner diameter of the distal end of the distal tip 14 is about 0.38 mm to about 0.40 mm.

In the embodiment illustrated in FIG. 2, the distal tip 14 is an integral part of the inner tubular member 18, i.e., the distal tip 14 and inner tubular member 18 are a one piece, single unit. In alternative embodiments, the distal tip is a separate member joined to the distal end of the catheter shaft. The distal tip 14 may be joined to the distal end of the catheter shaft using a variety of suitable means including adhesive bonding, fusing, and hot melt bonding. The internal taper 26 of the distal tip 14 may be formed before or after the distal tip 14 is joined to the distal end of the catheter shaft.

In a presently preferred embodiment, the distal tip 14 is formed of a polymeric material. The distal tip 14 may be a soft tip configured to provide an atraumatic distal end on the catheter to minimize injury to the patient's vasculature during advancement of the catheter therein. In one embodiment, the distal tip 14 is formed of a polymeric material having a lower Shore Durometer hardness than the polymeric material forming a section of the shaft 11 proximal thereto. A variety of polymeric materials may be used to form the tip 14 including polyamides such as Pebax (polyether block amide) and polyethylene based adhesives such as PRIMACOR, high density polyethylene (HDPE), polyurethane, and polyesters such as HYTREL. However, the choice of material depends on a variety of factors including the desired application and the method used to make the distal tip 14.

FIG. 5 illustrates one presently preferred embodiment of a method of making distal tip 14 using a mandrel having a tapered outer surface corresponding to the internal taper 26 of the distal tip 14. The method generally comprises placing at least a section of a polymeric tube 40 having a first end, a second end and a lumen therein on a mandrel 41 having at least a section with a tapered outer surface 42, heating the polymeric tube 40 so that the first end of the polymeric tube forms a tapered section having an inner surface tapering distally to an inner diameter smaller than an inner diameter of the polymeric tube closer to the second end of the polymeric tube. The embodiment illustrated in FIG. 5 also produces a tapered outer surface in the polymeric tube 40. Preferably, shrink tubing 43 is placed on the outer surface of polymeric tube 40 and heated, as by exposure to a laser, to apply heat and radially compressive pressure conforming the polymeric tube onto the mandrel tapered surface 42. Polymeric tube 40 may then be trimmed, joined to other catheter components, or otherwise processed as required to form the catheter 10 having distal tip 14. Presently preferred polymeric materials for forming distal tip 14 using the method illustrated in FIG. 5 are Pebax and polyurethane, having a Shore Durometer of about 40D to about 62D.

Alternatively, distal tip can be made using a straight mandrel. FIG. 6 illustrates another presently preferred method of making distal tip 14. The method generally comprises placing at least a section of a polymeric tube 50 having a first end, a second end and a lumen therein within a mold 51 having a section having a tapered inner surface 52. Polymeric tube 50 is on a mandrel 53 having a straight cylindrical surface so that a gap 54 is between an inner surface of the polymeric tube and an outer surface of the mandrel, and gap 55 is between an outer surface of the polymeric tube 50 and an inner surface of a section of the mold 51. The first end of the polymeric tube 50 is longitudinally displaced or urged into contact with the tapered end of the mold and heated, so that the first end of the polymeric tube forms a tapered section having an inner surface tapering distally to an inner diameter smaller than an inner diameter of the polymeric tube closer to the second end of the polymeric tube. The polymeric tube 50 having a tapered section thus formed is preferably cooled inside the mold before being removed form the mold. The embodiment illustrated in FIG. 6 also produces a tapered outer surface in the polymeric tube 50. In a presently preferred embodiment, mold 51 is heated to heat the polymeric tube 50. In the illustrated embodiment, a dam 56 comprising a shoulder defining a decreased inner diameter section is provided at the end of the mold. In alternative embodiments, mold 51 does not have dam 56, so that the mold tapered inner surface 52 extends to the end section of the mold having a straight inner surface generally parallel to the outer surface of the mandrel 53. A presently preferred polymeric material for forming distal tip 14 using the method illustrated in FIG. 6 is Pebax, having a Shore Durometer of about 55D to about 63D, although a variety of suitable thermoplastic materials including polyethylene and polyurethane may be formed into the distal tip 14 using the method of the invention.

As illustrated in FIG. 2, section 32 provides a section of the guidewire lumen having a smaller inner diameter and a length which is relatively short compared to the length of the inner tubular member 18. As a result, section 32 provides improved catheter centering and guidewire hugging without adversely affecting the longitudinal movement of the guidewire 24 within the guidewire lumen 19. Section 32 has a length substantially shorter than a length of the inner tubular member 19, i.e., not greater than about 1%, preferably not greater than about 0.04% to about 0.1% of the length of the inner tubular member 18. Section 32 has a length not greater than about 1.7% to about 6% of the length of the shaft extending beyond the inflatable interior 21 of the balloon 15. In one embodiment, the length of the section 32 is about 0.05 mm to about 0.12 mm, and the length of the internal taper 26 is about 0.12 mm to about 1 mm, and the length of the tapered outer surface of the distal tip 14 is about 0.12 mm to about 2 mm, preferably about 0.25 mm to about 1 mm. The length of the internal taper and the tapered outer surface will vary depending on the mold, mandrel and tip dimensions. In one embodiment, the internal taper tapers at an angle of about 14° to about 60°, more specifically about 14° to about 34° from the inner surface of the section of the inner tubular member 18 proximal to the distal tip. The tapered outer surface of the distal tip 14 tapers at an angle of about 14° to about 60°, more specifically about 14° to about 34° from the outer surface of the section of the inner tubular member 18 proximal to the distal tip. The inner diameter of the distal end of the distal tip 14 is about 3% to about 20% smaller than the inner diameter of the section of the inner tubular member 18 proximal to the distal tip.

FIG. 7 illustrates an alternative embodiment of the invention, having a sleeve 60 on an outer surface of a proximal section of the distal tip 14. In the embodiment illustrated in FIG. 7, distal tip 14 is a separate member joined to the distal end of the catheter shaft inner tubular member 18. Sleeve 60 extends over the both the distal tip 14 and the inner tubular member 18 and the junction therebetween. The sleeve 60 may be secured to the distal tip 14 and the inner tubular member 18 by a variety of suitable methods, including fusion bonding and adhesive bonding. In one embodiment, sleeve 60 is formed of a polymeric material compatible and fusible with the polymeric material of the distal tip 14 and/or the inner tubular member 18. Sleeve 60 has a tapered outer surface 61 which is aligned with the tapered outer surface 29 of the distal tip 14. Thus, the distal ends of the distal tip 14 and the sleeve 60 taper at the same angle. In the embodiment illustrated in FIG. 7, a proximal section of the sleeve 60 has an outer surface which is generally parallel with the outer surface of the inner tubular member, so that the proximal end of the tapered outer surface 61 of the sleeve 60 is located distal to the proximal end of the sleeve 60. Proximal end of the sleeve 60 is adjacent to and may be secured to the distal balloon shaft.

A method of making the distal tip 14 illustrated in FIG. 7 having a sleeve 60 with a tapered outer surface 61 tapering in alignment with the distal tip outer surface is illustrated in FIG. 8. The embodiment illustrated in FIG. 8 is similar to the embodiment illustrated in FIG. 6 having a straight mandrel 53 in a mold 51, except that in the embodiment illustrated in FIG. 8, mold 51 has a tapered inner surface 58 which extends a substantial length of the mold 51, compared to tapered inner surface 52 of the embodiment illustrated in FIG. 6. As a result, sleeve 60 on an outer surface of the polymeric tubular member 50 which is formed into the distal tip 14 is longitudinally displaced or urged into contact with the tapered surface 58 of the mold 51 and heated along with the first end of the polymeric tube 50, so that the first end of the polymeric tube 50 forms a tapered section having an inner surface tapering distally to an inner diameter smaller than an inner diameter of the polymeric tube closer to the second end of the polymeric tube, and the outer surfaces of at least a section of the polymeric tube 50 and the sleeve 60 are tapered, preferably at the same angle, to thereby form the tapered outer surface 29 of the distal tip 14 and the tapered outer surface 61 of the sleeve 60. Alternatively, depending on factors such as the relative sizes of the polymeric tube 50, mandrel 53 and mold 51, the method illustrated in FIG. 8 can be used to prepare a distal tip having a tapered outer surface 29 and with a sleeve 60 thereon having a tapered outer surface 61, wherein the distal tip 14 does not have a tapered inner surface (not shown). In the embodiment illustrated in FIG. 8, sleeve 60 is bonded to the distal tip 14 by a heat fusion bond, and as a result of the fusion bonding process, the sleeve already has a slightly tapered outer surface when it is placed into the mold 51 for performing the distal tip forming method of the invention. In an alternative embodiment, the sleeve has a straight outer surface generally parallel to the surface of the polymeric tube 50. A junction between the distal tip 14 and the inner tubular member 18 is proximal to the section of the distal tip 14 illustrated in FIG. 8, and is therefore not illustrated in FIG. 8.

The dimensions of catheter 10 are determined largely by the size of the guidewires to be employed and the size of the artery or other body lumen through which the catheter must pass or the size of the stent being delivered. Typically, the outer tubular member 14 has an outer diameter of about 0.02 to about 0.04 inch (0.05 to 0.10 cm), usually about 0.037 inch (0.094 cm), an inner diameter of about 0.015 to about 0.035 inch (0.038 to 0.089 cm), usually about 0.03 inch (0.076 cm). The wall thickness of the outer tubular member 16 can vary from about 0.002 to about 0.008 inch (0.0051 to 0.0201 cm), typically about 0.003 inch (0.0076 cm). The inner tubular member 18 typically has an outer diameter of about 0.019 to about 0.028 inch, usually about 0.021 inch. The overall working length of the catheter 10 may range from about 100 to about 150 cm, and is typically about 147 cm. Preferably, balloon 15 may have a length about 0.5 cm to about 4 cm and typically about 2 cm with an inflated working diameter of about 1 to about 8 mm, and for coronary applications about 1.5 mm to about 5 mm.

To the extent not discussed herein, the various catheter components can be formed of conventional materials. Inner tubular member 18 and outer tubular member 16 can be formed by conventional techniques, for example by extruding, from materials already found useful in intravascular catheters such a polyethylene, polyvinyl chloride, polyesters, polyamides, polyimides and composite materials. The various components may be joined by heat bonding or use of adhesives.

A variety of suitable catheter designs may be used, including rapid exchange, over-the-wire, and fixed wire catheter designs. A rapid exchange catheter generally includes an inflation lumen extending from the proximal end of the catheter shaft to a location spaced proximal to the distal end of the catheter shaft, a distal guidewire port in the distal end of the catheter shaft, a proximal guidewire port spaced distal to the proximal end of the catheter shaft, and a guidewire lumen extending between the proximal and distal guidewire ports. Typically, the proximal guidewire port is spaced a substantial distance from the proximal end of the catheter shaft and a relatively short distance from the distal guidewire port, so that the proximal guidewire port is closer to the distal guidewire port than to the proximal end of the catheter shaft.

Although not illustrated, the balloon catheter of the invention may be used to deliver prostheses, such as expandable stents, grafts, and the like, to a desired location within the patient's vasculature. A stent (not shown) comprising an expandable tubular body, typically having an open-walled structure, may be mounted on balloon 15, and balloon 15 may be inflated to expand the stent and seat it in the vessel. Additionally, catheter 10 may be used to touch up a previously implanted stent by positioning balloon within stent lumen and expanding the balloon to further expand the stent within a body lumen.

While the present invention is described herein in terms of certain preferred embodiments, those skilled in the art will recognize that various modifications and improvements may be made to the invention without departing from the scope thereof. For example, although discussed primarily in terms of a tapered distal tip having an internal taper and a tapered outer surface, the internal taper of the invention may be located in other sections of the catheter shaft. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

What is claimed is:

1. An intraluminal catheter, comprising an elongated shaft having a proximal end, a distal end, at least one lumen, and a distal tip having an internal taper.

2. The catheter of claim 1 wherein the distal tip internal taper is at the distal end of the distal tip.

3. The catheter of claim 1 wherein the distal tip has an outer surface tapering distally.

4. The catheter of claim 3 wherein the tapered outer surface of the distal tip tapers distally to the distal end of the shaft.

5. The catheter of claim 3 wherein the proximal end of the tapered outer surface of the distal tip is radially aligned with a proximal end of the internal taper.

6. The catheter of claim 3 wherein a distal end of the internal taper is longitudinally aligned with a distal end of the tapered outer surface of the distal tip.

7. The catheter of claim 1 wherein the distal tip is an integral part of the elongated shaft.

8. The catheter of claim 1 wherein the distal tip is a separate member joined to the elongated shaft.

9. The catheter of claim 1 wherein the distal tip is formed of a polymeric material.

10. The catheter of claim 1 wherein the distal tip is formed of a material having a lower Shore Durometer hardness than a proximal section of the elongated shaft.

11. The catheter of claim 1 wherein the catheter is a balloon catheter including a balloon on a distal portion of the shaft.

12. The catheter of claim 11 wherein the distal tip is distal to the proximal end of the balloon.

13. The catheter of claim 3 wherein the tapered outer surface of the distal tip has a length of about 0.12 mm to about 5 mm.

14. The catheter of claim 1 wherein the internal taper has a length of about 0.12 mm to about 5 mm.

15. The catheter of claim 3 wherein the tapered outer surface of the distal tip tapers at an angle of about 14 to about 60 degrees from the outer surface of the shaft proximal to the distal tip.

16. The catheter of claim 1 wherein the internal taper tapers at an angle of about 14 to about 60 degrees from an inner surface of the shaft proximal to the distal tip.

17. The catheter of claim 1 wherein a lumen in a distal end of the distal tip is about 3% to about 20% smaller than a lumen in the shaft proximal to the distal tip.

18. A balloon catheter, comprising
a) an elongated shaft having a proximal end, a distal end, an inflation lumen, a guidewire lumen, and a distal tip having an internal taper; and
b) a balloon secured to a distal portion of the catheter shaft having an inflatable interior which is in fluid communication with the inflation lumen.

19. The catheter of claim 18 wherein the balloon inflatable interior is proximal to the distal tip.

20. The catheter of claim 18 wherein the catheter shaft comprises an outer tubular member defining the inflation lumen, and an inner tubular member disposed within the outer tubular member and defining the guidewire lumen, and wherein the distal tip is at the distal end of the inner tubular member.

21. The catheter of claim 20 wherein the inner tubular member distal tip has an outer surface tapering distally to smaller outer diameter.

22. The catheter of claim 20 including a guidewire slidably disposed within the guidewire lumen, the distal end of the guidewire lumen having an inner diameter configured to allow longitudinal displacement of the guidewire therein and to restrain radial displacement of the guidewire therein.

23. The catheter of claim 22 wherein the inner diameter of the distal end of the guidewire lumen is about 3% to about 14% larger than an outer diameter of the guidewire.

24. A method of making a tapered section for a catheter shaft, comprising
a) placing at least a section of a polymeric tube having a first end, a second end and a lumen therein within a mold having a tapered inner surface along at least a section of an inner surface of the mold and on a mandrel having a straight cylindrical surface so that a gap is between an inner surface of the polymeric tube and an outer surface of the mandrel;
b) placing the first end of the polymeric tube in contact with the tapered inner surface of the mold and heating the polymeric tube, so that a tapered section is formed at the first end of the polymeric tube having an inner surface tapering to a smaller inner diameter toward the first end of the polymeric tube.

25. The method of claim 24 including urging the polymeric tube against the tapered end of the mold.

26. The method of claim 24 including forming tapered outer surface of the polymeric tube tapering to a smaller outer diameter toward the first end.

27. The method of claim 26 wherein the polymeric tube includes a sleeve on at least a section of an outer surface thereof, and including placing a first end of the sleeve in contact with the tapered end of the mold and heating the sleeve, so that the first end of the sleeve has an outer surface tapering to a smaller outer diameter toward the first end and aligned with the tapered outer surface of the polymeric tube.

28. A method of making a tapered section for a catheter shaft, comprising
a) placing at least a section of a polymeric tube having a first end, a second end, a lumen therein, and a sleeve secured to at least a section of an outer surface of the polymeric tube on a mandrel having a straight cylindrical surface and within a mold having a tapered inner surface along at least a section of the mold; and
b) placing the first end of the polymeric tube and a first end of the sleeve in contact with the tapered inner surface of the mold and heating the polymeric tube and sleeve, so that a tapered section on the sleeve is formed having a tapered outer surface tapering to a smaller outer diameter toward the first end of the polymeric tube, and a tapered section on the polymeric tube is formed having a tapered outer surface aligned with the tapered outer surface of the sleeve and tapering to a smaller outer diameter toward the first end of the polymeric tube.

29. A balloon catheter, comprising
a) an elongated shaft having a proximal end, a distal end, an inflation lumen, a guidewire lumen, a distal tip, and a sleeve secured to at least a section of the shaft and at least a section of the distal tip, the sleeve having at least a section with a tapered outer surface tapering to a smaller outer diameter toward the distal end of the shaft, and the distal tip having at least a section with a tapered outer surface aligned with the tapered outer surface of the sleeve and tapering to a smaller outer diameter toward the distal end of the shaft; and
b) a balloon secured to a distal portion of the catheter shaft having an inflatable interior which is in fluid communication with the inflation lumen.
